(19) United States
(12) Patent Application Publication
(10) Pub. No.: US 2017/0241831 A1
(43) Pub. Date: Aug. 24, 2017

(54) Enhanced Acoustic Sensing System

(71) Applicant: Halliburton Energy Services, Inc, Houston, TX (US)

(72) Inventor: Mikko Jaaskelainen, Katy, TX (US)

(21) Appl. No.: 15/506,092

(22) PCT Filed: Oct. 9, 2014
(86) PCT No.: PCT/US14/59842

§ 371 Date: Feb. 23, 2017

(51) Int. Cl. G01H 9/00; A61B 7/02; A61B 5/00; G01N 29/24

(52) U.S. Cl. CPC G01H 9/004; G01N 29/2418; A61B 7/02; A61B 5/0095

(57) ABSTRACT

A system and method for enhanced acoustic sensing of signals in a pipe using a fiber optic sensing cable that can be interrogated by distributed acoustic sensing (DAS) systems wherein a crescent shaped metallic device is used for attaching to the exterior of the pipe, the crescent shaped metallic device having the one or more fiber optic sensing cables embedded within an upper part of the crescent shaped device, and the crescent shaped metallic device may have one or more cavities or channels that may be empty or partially or completely filled with acoustic filters.

### BACKGROUND

This disclosure relates generally to acoustic sensing, and more particularly, to acoustic sensing systems for various types of piping which might include tubing, casing, flow lines, pipe lines etc., in such systems where the signals are concentrated and optimally coupled to a fiber optic sensing cable that can be interrogated using e.g. Distributed Acoustic Sensing (DAS) systems.

Fiber optic sensing cables are deployed on pipes (tubing, casing, flow lines, pipe lines etc.) today, and the optical fibers are connected to interrogation units like e.g. coherent Rayleigh based Distributed Acoustic Sensing (DAS) systems and/or Distributed Temperature Sensing (DTS) systems. Acoustic energy is transmitted to the cable, and optical fibers, and this acoustic energy can be used to determine e.g. flow rates inside the pipes. The fiber optic cables are commonly strapped outside the pipe.

One of the challenges with the systems currently in use is the coupling from the pipe to the cable housing the fibers. The sensing cables are normally in contact with the pipe, but the contact area is very small, and the sensitivity of the system suffers, which in turn may make the measurements noisy and in some cases not possible.

There is a need then for a technique or method to enhance the sensitivity and performance of these systems.

### BRIEF DESCRIPTION OF THE DRAWINGS

FIG. 1 illustrates a commonly used approach of attaching a sensing cable to a pipe.

FIG. 2 illustrates a device with enhanced acoustic coupling between pipe and sensing cable.

FIG. 3 illustrates the analogy of the use of a stethoscope to collect acoustic energy.

FIG. 4 illustrates a device with enhanced acoustic coupling between pipe and sensing cable using a cavity and membrane.

FIG. 5 illustrates a device making use of enhanced acoustic coupling combined with an acoustic filter.

### DETAILED DESCRIPTION

In the following detailed description, reference is made to accompanying drawings that illustrate embodiments of the present invention. These embodiments are described in sufficient detail to enable a person of ordinary skill in the art to practice the invention without undue experimentation. It should be understood, however, that the embodiments and examples described herein are given by way of illustration only, and not by way of limitation. Various substitutions, modifications, additions, and rearrangements may be made without departing from the spirit of the present invention. Therefore, the description that follows is not to be taken in a limited sense, and the scope of the present invention will be defined only by the final claims.

Optical fibers are often deployed within fiber optic sensing cables which are deployed on pipes (tubing, casing, flow lines, pipe lines etc.) today, and the optical fibers are connected to interrogation units like e.g. coherent Rayleigh based Distributed Acoustic Sensing (DAS) systems and/or Distributed Temperature Sensing (DTS) systems. Acoustic energy is transmitted to the cable, and optical fibers, and this acoustic energy can be used to determine e.g. flow rates inside the pipes. The fiber optic cables are commonly deployed by being strapped outside the pipe.

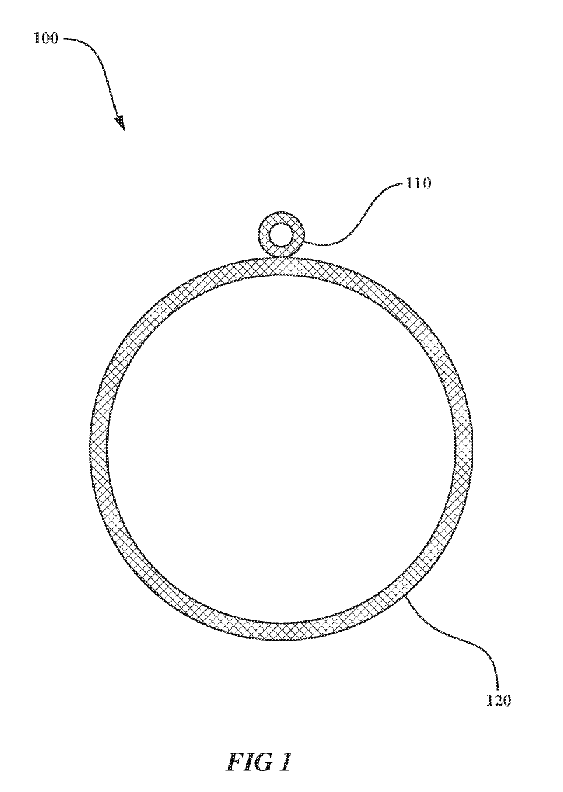

FIG. 1, shown generally as the numeral 100, illustrates a commonly used contact principle between a pipe 120, and an acoustic cable 110 in contact with the pipe. One of the challenges with such systems is the coupling from the pipe to the cable housing the fibers. The sensing cables are normally in contact with the pipe, but the contact area is very small, and the sensitivity of the system suffers, which in turn may make the measurements noisy and in some cases not possible.

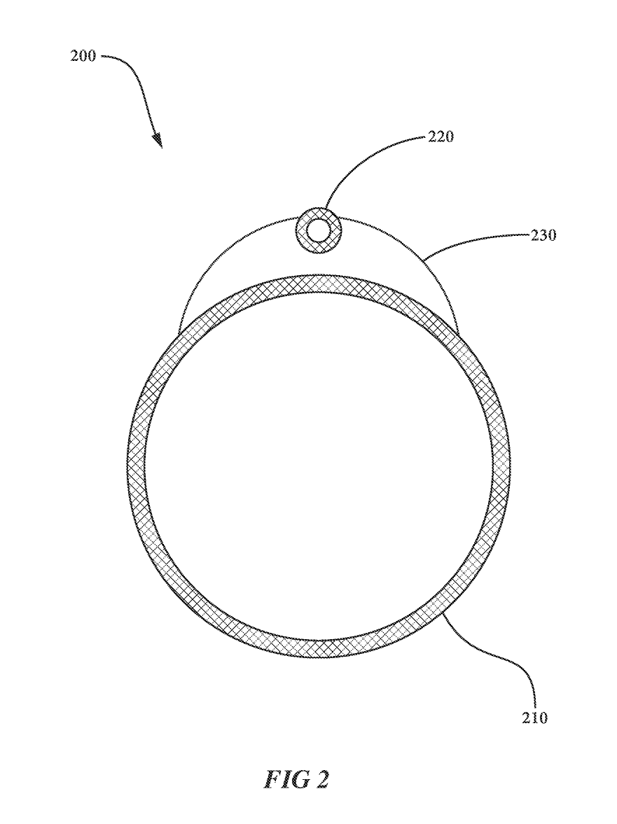

One approach to changing this reality is an enhanced system as shown in FIG. 2, illustrated by the numeral 200. This enhanced system is shown as one embodiment in FIG. 2, in which a fiber optic sensing cable 220 is embedded in a device 230 that is shaped to have a dramatically larger contact area with respect to the pipe, thus improving the path for the acoustic energy to reach the sensing cable. Device 230 is made in a crescent shape that can be tightly clamped or attached along a length of pipe 210 to greatly increase the contact area for picking up acoustic information from the pipe. The fiber optic sensing cable would normally be embedded in the upper part of crescent 230 and the lower part of the crescent would be shaped to be in intimate contact with pipe 210. The crescent shaped device could be applied in a long continuous fashion lengthwise on the pipe or applied along a plurality of sensing positions along the pipe. The application anticipates either of these or combinations.

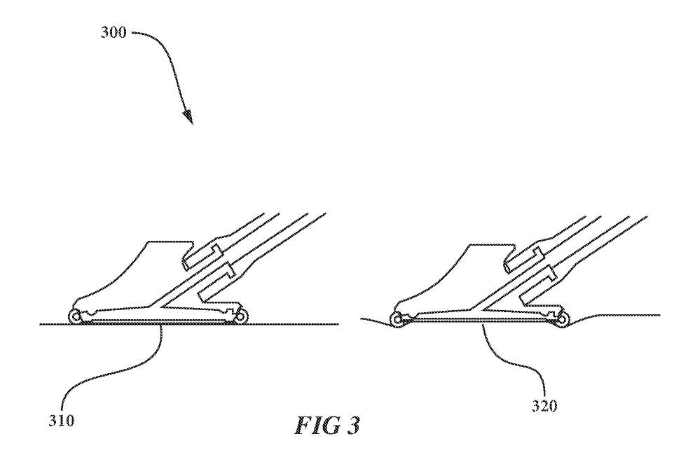

The field of stethoscopes offers an approach for further enhancing the acoustic coupling between a pipe and the sensing cable. Stethoscopes are widely used and are in essence a mechanical amplifier/collector of acoustic energy. For example, FIG. 3, illustrated generally by the numeral 300, illustrates how one type of stethoscope can be used to listen for both low and higher frequency sounds. In example 310 (Bell Mode) a doctor can use light contact with a chest piece and listen for low-frequency sounds, in this bell mode the vibrations of the skin directly produce acoustic pressure waves traveling up to the listener's ears. In example 320 (diaphragm mode) much more pressure is used, pressuring the device down onto the skin, and the device becomes much more sensitive to higher frequency body sounds. In both modes the air cavity acts to gather the acoustic energy and transmit it up the air tubes into the doctors ears.

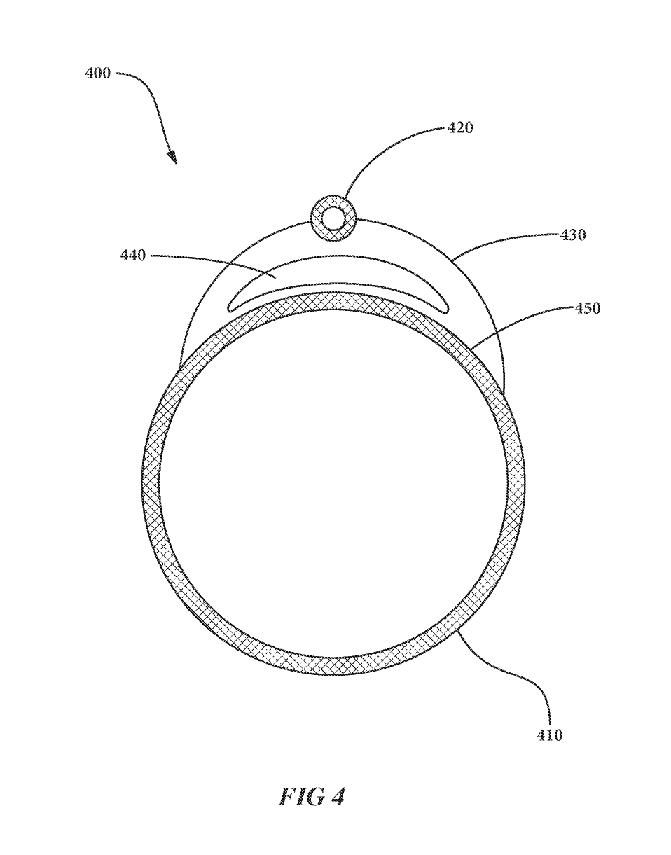

FIG. 4, shown generally by the numeral 400, illustrates another proposed crescent shaped device 430 with enhanced acoustic coupling between pipe 410 and sensing cable 420 that now includes a cavity 440 and a membrane portion 450 that is in intimate contact along the length of pipe 410. Device 430 is a crescent shaped piece that is again shaped to have intimate contact with a length of pipe 410 along membrane 450. The fiber optic sensing cable 420 is embedded into device 430. In a manner similar to the stethoscope described earlier the extended cavity stretched over an extended piece of the membrane in intimate contact along a length of pipe helps to gather acoustic energy that is transmitted into device 430 and thus into fiber optic sensing cable 420. Alternate combinations of cavity size and membrane thickness can be optimized for different desired frequencies. In addition there can be one or more cavities or channels on either side (not shown). These can provide channels with different acoustic impedance (e.g. air) directing energy towards the sensing cable.

The device will be shaped to couple closely with the pipe and the fiber optic sensing cable, and a compound with suitable acoustic properties can be used at the interfaces between the membrane and pipe and between the fiber optic sensing cable 420 and device 430 to ensure good coupling.

This disclosure assumes any number of suitable materials of construction for device 430. Some desired options could be Inconel 718, Inconel 625, Titanium TI64, Cobalt Chrome, Stainless Steel 17-4 PH, Alloy 825, or Kovar nickel-cobalt ferrous alloy.

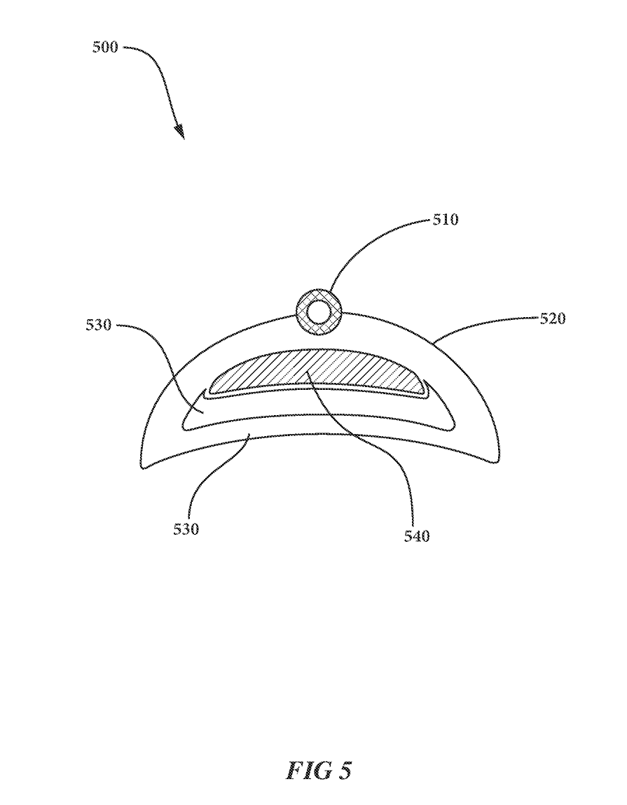

The device of FIG. 4 can be further enhanced by the embodiment shown in FIG. 5, shown generally by the numeral 500. In this embodiment a fiber optic sensing cable 510 is again embedded into a crescent shaped device 520 and again includes a cavity 530, but also includes an acoustic filter 540 to block chosen noise bands based on the application. It is known that fluids in general have lower frequency content than gases, and sand/frac proppants/solids may have yet another frequency characteristics. The design of FIG. 5 can be used to combine the type of acoustic filter with the cavity size to provide good acoustic sensitivity for desired frequencies and to screen out the known undesired frequencies. In some embodiments the acoustic filter may completely fill the entire cavity.

In use any of the proposed systems could operate by transmitting a light pulse (or light pulses) through the optical fibers within the one or more fiber optic sensing cables; interrogating coherent Rayleigh backscatter signals generated by the transmission of the light pulse(s) and acoustic and/or vibration signals; processing the coherent Rayleigh signals to identify acoustic occurrences along the pipe; and embedding the one or more fiber optic sensing cables in a crescent shaped metallic device for attaching to the exterior of the pipe.

Although certain embodiments and their advantages have been described herein in detail, it should be understood that various changes, substitutions and alterations could be made without departing from the coverage as defined by the appended claims. Moreover, the potential applications of the disclosed techniques is not intended to be limited to the particular embodiments of the processes, machines, manufactures, means, methods and steps described herein. As a person of ordinary skill in the art will readily appreciate from this disclosure, other processes, machines, manufactures, means, methods, or steps, presently existing or later to be developed that perform substantially the same function or achieve substantially the same result as the corresponding embodiments described herein may be utilized. Accordingly, the appended claims are intended to include within their scope such processes, machines, manufactures, means, methods or steps.

1. A system for enhanced acoustic sensing of signals in a pipe using optical fibers within one or more fiber optic sensing cables that can be interrogated by distributed acoustic sensing (DAS) systems comprising:
a. one or more light sources for introducing light into the optical fibers;
b. an interrogator unit for interrogating the backscattered light from optical fibers deployed within the fiber optic sensing cable;
c. a processor for analyzing the acoustic signals detected; and
d. a crescent shaped metallic device for attaching to the exterior of the pipe, the crescent shaped metallic device having the one or more fiber optic sensing cables embedded within an upper part of the crescent shaped device.

2. The system for enhanced acoustic sensing of signals in a pipe using optical fibers within a fiber optic sensing cable that can be interrogated by distributed acoustic sensing (DAS) systems of claim 1 wherein the crescent shaped metallic device includes one or more empty cavities between the upper part of the crescent shaped device containing the one or more fiber optic sensing cables and a bottom metallic membrane of the crescent shaped metallic device attached against the pipe.

3. The system for enhanced acoustic sensing of signals in a pipe using optical fibers within a fiber optic sensing cable that can be interrogated by distributed acoustic sensing (DAS) systems of claim 2 wherein there is one central empty cavity and it is also crescent shaped.

4. The system for enhanced acoustic sensing of signals in a pipe using optical fibers within a fiber optic sensing cable that can be interrogated by distributed acoustic sensing (DAS) systems of claim 2 wherein acoustic filters designed to filter chosen acoustic frequencies are located in the one or more empty cavities.

5. The system for enhanced acoustic sensing of signals in a pipe using optical fibers within a fiber optic sensing cable that can be interrogated by distributed acoustic sensing (DAS) systems of claim 4 wherein acoustic filters completely fill the one or more central cavities.

6. The system for enhanced acoustic sensing of signals in a pipe using optical fibers within a fiber optic sensing cable that can be interrogated by distributed acoustic sensing (DAS) systems of claim 1 wherein the one or more light sources for introducing light comprises pulsed lasers.

7. A method for enhanced acoustic sensing of signals in a pipe using optical fibers within one or more fiber optic sensing cables that can be interrogated by distributed acoustic sensing (DAS) systems comprising:
a. transmitting a light pulse through the optical fibers within the one or more fiber optic sensing cables;
b. interrogating coherent Rayleigh signals generated by the transmission of the light source;
c. processing the coherent Rayleigh signals to identify acoustic occurrences along the pipe; and
d. embedding the one or more fiber optic sensing cables in a crescent shaped metallic device for attaching to the exterior of the pipe.

8. The method for enhanced acoustic sensing of signals in a pipe using optical fibers within one or more fiber optic sensing cables that can be interrogated by distributed acoustic sensing (DAS) systems of claim 7 wherein the crescent shaped metallic device includes one or more empty cavities between the upper part of the crescent shaped device containing the one or more fiber optic sensing cables and a bottom metallic membrane of the crescent shaped metallic device attached against the pipe.

9. The method for enhanced acoustic sensing of signals in a pipe using optical fibers within one or more fiber optic sensing cables that can be interrogated by distributed acoustic sensing (DAS) systems of claim 7 wherein there is one central empty cavity and it is also crescent shaped.

10. The method for enhanced acoustic sensing of signals in a pipe using optical fibers within one or more fiber optic sensing cables that can be interrogated by distributed acoustic sensing (DAS) systems of claim 9 wherein acoustic filters designed to filter chosen acoustic frequencies are located in the one or more empty cavities.

11. The method for enhanced acoustic sensing of signals in a pipe using optical fibers within one or more fiber optic sensing cables that can be interrogated by distributed acoustic sensing (DAS) systems of claim 10 wherein acoustic filters fill the one or more central cavities.

12. The method for enhanced acoustic sensing of signals in a pipe using optical fibers within one or more fiber optic sensing cables that can be interrogated by distributed acoustic sensing (DAS) systems of claim 7 wherein the light source for introducing light comprises a pulsed laser.
